(19) United States
(12) Patent Application Publication
(10) Pub. No.: US 2012/0077803 A1
(43) Pub. Date: Mar. 29, 2012

(54) Uses Of NK Receptor Antagonists

(75) Inventors: Anton Stuetz, Wien (AT); Barbara Wolff-Winiski, Wien (AT); Lina Williamson, Boston, MA (US)

(73) Assignee: NOVARTIS AG, Basel (CH)

(21) Appl. No.: 13/202,741

(22) PCT Filed: Feb. 23, 2010
(86) PCT No.: PCT/EP10/52273

§ 371 Date: Dec. 1, 2011

Related U.S. Application Data
(60) Provisional application No. 61/154,973, filed on Feb. 24, 2009.

(51) Int. Cl. A61K 31/55; A61P 17/00; A61P 35/00; A61P 17/06; A61P 17/04

(52) U.S. Cl. 514/212.03

(57) ABSTRACT

The invention provides the use of a compound of formula (I)

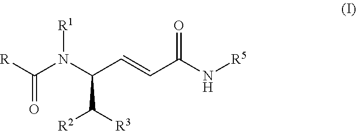

or a solvate or hydrate thereof, wherein the substituents are as defined in the description, and combinations thereof, for the treatment of pruritus or a dermatological disorder or disease.

The present invention relates to uses of antagonists with activity for the neurokinin 1 (NK1) receptor, for both the NK1 and NK2 receptors, and/or for all three NK1, NK2 and NK3 receptors. The antagonists are acylaminoalkenylene-amide derivatives of formula (I), for use in the treatment of pruritus or a dermatological disorder or disease. The invention also relates to pharmaceutical compositions for such uses and to combinations for such uses.

WO98/07694 describes acylaminoalkenylene-amide derivatives and their medical uses, particularly in the treatment of a number of conditions associated with substance P and neurokinin. WO2007/118651 describes preparation of acylaminoalkenylene-amide derivatives as depicted in formula (I). The use of a compound of formula (I) in the treatment of pruritus or a dermatological disorder or disease is not disclosed therein.

There are unsatisfactory currently available therapeutic options to treat pruritus. Creams or lotions are available, for example containing capsaicin, menthol, camphor, urea, polidocanol or tannin, but these bring only temporary relief. Calcineurin antagonists have been shown to reduce pruritus, and corticosteroids are directed to treating the underlying dermatological disease. Antihistamines have a very limited effectiveness. Anticonvulsants, antidepressants and antiserotoninergic substances as well as opioid antagonists are used, but have severe side effects. There is a high unmet medical need for compounds that provide an effective treatment of pruritus.

In a first aspect the present invention provides a compound of formula (I)

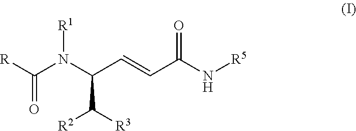

or a solvate or hydrate thereof, where
R is phenyl that is unsubstituted or is substituted by 1, 2 or 3 substituents selected from the group consisting of halogen, C₁-C₇-alkyl, trifluoromethyl, hydroxy and C₁-C₇-alkoxy;
R¹ is hydrogen or C₁-C₇-alkyl;
R² is hydrogen C₁-C₇-alkyl or phenyl that is unsubstituted or is substituted by 1, 2 or 3 substituents selected from the group consisting of halogen, C₁-C₇-alkyl, trifluoromethyl, hydroxy and C₁-C₇-alkoxy;
R³ is phenyl that is unsubstituted or is substituted by 1, 2 or 3 substituents selected from the group consisting of halogen, C₁-C₇-alkyl, trifluoromethyl, hydroxy and C₁-C₇-alkoxy, or R³ is naphthyl, 1H-indol-3-yl or 1-C1-C7-alkyl-indol-3-yl; and
R⁵ is C₃-C₈-cycloalkyl, D-azacycloheptan-2-on-3-yl or L-azacycloheptan-2-on-3-yl;
   for the treatment of pruritus. The compound of formula (I) is also referred to as the agent of the invention.

The invention, including embodiments thereof as described herein, provides a compound of formula (I), or a solvate or hydrate thereof, for the treatment of pruritus or a dermatological disorder or disease, having one or more of the following unexpected benefits:
prolonged relief for the patient, for example being useful in the treatment of pruritus present over an extended period of time;
a particularly favourable efficacy profile;
a particularly favourable tolerability and side effect profile, for example with respect to central nervous system side effects;
an anti-inflammatory effect, for example in the skin;

Central nervous system side effects include headache, fatigue, insomnia and nausea.

The invention may be more fully appreciated by reference to the following description, including the following glossary of terms and the concluding examples.

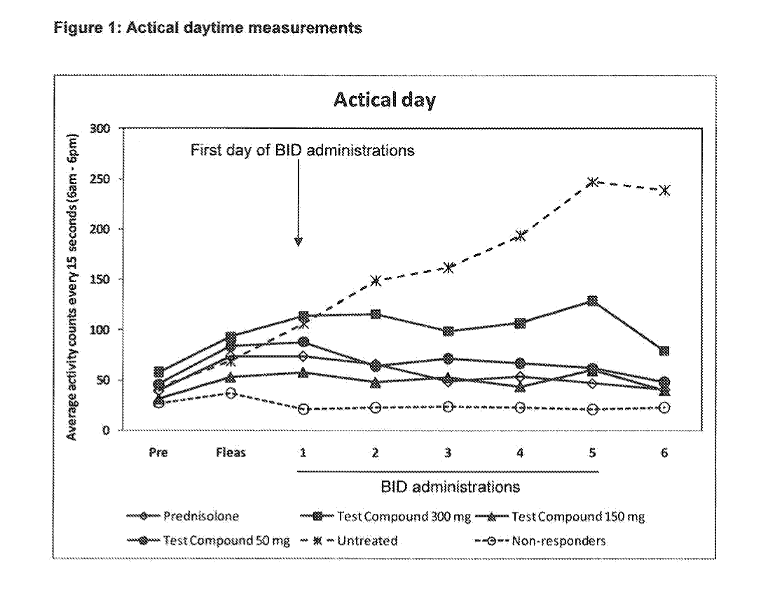

FIG. 1 discloses the daytime activity measurements of flea allergic dogs, following treatment to evaluate the activity of a test compound of formula (I).

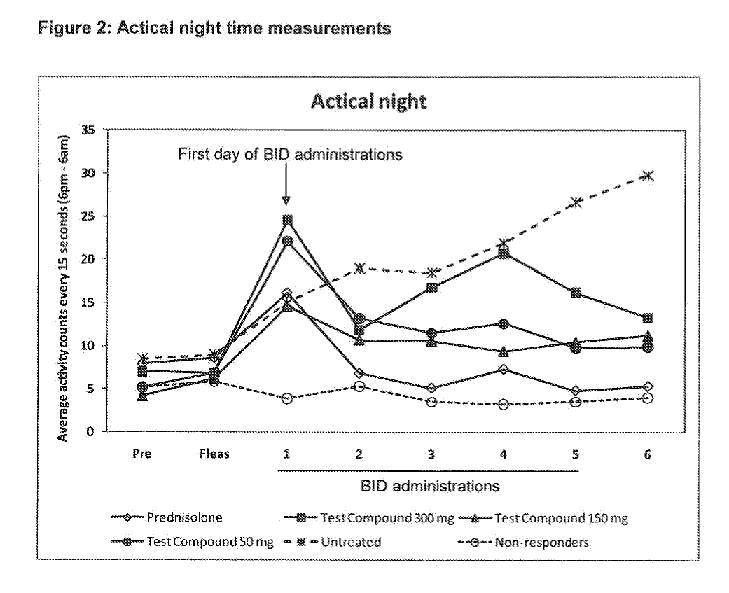

FIG. 2 discloses the night time activity measurements of flea allergic dogs, following treatment to evaluate the activity of a test compound of formula (I).

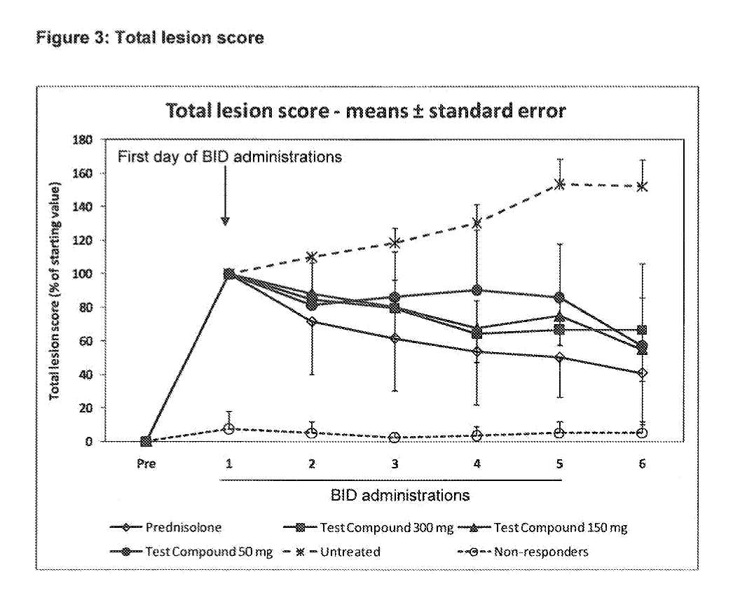

FIG. 3 discloses the total lesion scores of flea allergic dogs, following treatment to evaluate the activity of a test compound of formula (I).

As used herein, the terms “including”, “containing” and “comprising” are used herein in their open, non-limiting sense.

Any formula given herein is intended to represent compounds having structures depicted by the structural formula as well as certain variations or forms. In particular, compounds of any formula given herein may have asymmetric centers and therefore exist in different enantiomeric forms. If at least one asymmetrical carbon atom is present in a compound of the formula (I), such a compound may exist in optically active form or in the form of a mixture of optical isomers, e.g. in the form of a racemic mixture. All optical isomers and their mixtures, including the racemic mixtures, are part of the present invention. Thus, any given formula given herein is intended to represent a racemate, one or more enantiomeric forms, one or more diastereomeric forms, one or more atropisomeric forms, and mixtures thereof. Furthermore, certain structures may exist as geometric isomers (i.e. cis and trans isomers), as tautomers, or as atropisomers.

Any formula given herein is intended to optionally include hydrates, solvates, polymorphs of such compounds, and mixtures thereof.

For any formula given herein, any pharmaceutically acceptable salt thereof is also optionally encompassed.

When referring to any formula given herein, the selection of a particular moiety from a list of possible species for a specified variable is not intended to define the moiety for the variable appearing elsewhere. In other words, where a variable appears more than once, the choice of the species from a specified list is independent of the choice of the species for the same variable elsewhere in the formula (where one or more, up to all, general expressions in embodiments characterized as preferred above or below can be replaced with a more specific definition, thus leading to a more preferred embodiment of the invention, respectively). The same applies to other definitions, such as disorders and their preferences.

Where the plural form (e.g. compounds, hydrates) is used, this includes the singular (e.g. a single compound, a single hydrate). “A compound” does not exclude that (e.g. in a pharmaceutical formulation) more than one compound of the formula (I) (or a hydrate thereof) is present.

“Halogen” or “halo” as used herein denotes a element belonging to group 17 (formerly group VII) of the Periodic Table of Elements, which may be, for example, fluorine, chlorine, bromine or iodine. Preferably halogen or halo is chlorine or bromine, especially chlorine.

“C₁-C₇-alkyl” as used herein denotes a straight chain or branched alkyl group comprising 1 to 7 carbons, which may be, for example, methyl, ethyl, n-propyl, isopropyl, n-butyl, isobutyl, sec-butyl, tert-butyl, straight or branched pentyl, straight or branched hexyl or straight, or branched heptyl.

“C₁-C₇-alkoxy” as used herein denotes a straight chain or branched alkyl chain linked to O, which may be, for example, methoxy, ethoxy, n-propoxy, isopropoxy, n-butoxy, isobutoxy, sec-butoxy, tert-butoxy, straight or branched pentoxy, straight or branched hexyloxy, or straight or branched heptyloxy.

“C₃-C₈-cycloalkyl” as used herein denotes a fully saturated carbocyclic ring having 3 to 8 ring carbon atoms, for example a monocyclic group such as a cyclopropyl, cyclobutyl, cyclopentyl, cyclohexyl, cycloheptyl or cyclooctyl, or a bicyclic group such as bicycloheptyl or bicyclooctyl.

Compounds of formula (I) or intermediate compounds that are used to prepare compounds of formula (I) can be pharmaceutically acceptable isotopically-labelled compounds of formula (I) or isotopically-labelled intermediate compounds that are used to prepare compounds of formula (I) respectively wherein one or more atoms are replaced by atoms having the same atomic number, but an atomic mass or mass number different from the atomic mass or mass number usually found in nature. Examples of isotopes suitable for inclusion in the compounds of the invention include isotopes of hydrogen e.g. 2H and 3H, carbon e.g. 11C, 13C and 14C, chlorine e.g. 36Cl, fluorine e.g. 18F, iodine e.g. 123I and 125I, nitrogen e.g. 13N and 15N, oxygen e.g. 15O, 17O and 18O, and sulfur e.g. 35S. Certain isotopically-labelled compounds of formula (I), for example those incorporating a radioactive isotope, are useful in drug and/or substrate tissue distribution studies. The radioactive isotopes tritium (3H) and carbon-14 (14C) are particularly useful for this purpose in view of their ease of incorporation and ready means of detection. Substitution with heavier isotopes such as deuterium (2H) may afford certain therapeutic advantages that result from greater metabolic stability, for example increased in vivo half-life or reduced dosage requirements, and hence may be preferred in some circumstances. Substitution with positron emitting isotopes, such as 11C, 18F, 15O, and 13N can be useful in Positron Emission Topography (PET) studies for examining substrate receptor occupancy. Isotopically-labelling compounds of formula (I) or isotopically-labelling compounds of intermediate compounds that are used to prepare compounds of formula (I) can generally be achieved by conventional techniques known to those skilled in the art or by processes analogous to those described in the accompanying examples using an appropriate isotopically-labelled reagent in place of the non-labelled reagent previously used. Pharmaceutically acceptable solvates in accordance with the invention include those wherein the solvent of crystallisation may be isotopically substituted e.g. D2O, d6-acetone or d6-DMSO.

The term “combination” as used herein refers to either a fixed combination in one dosage unit form, or a kit of parts for the combined administration where a compound of the formula (I) and a combination partner (e.g. an other drug as explained below, also referred to as “therapeutic agent” or “co-agent”) may be administered independently at the same time or separately within time intervals, especially where these time intervals allow that the combination partners show a cooperative, e.g. synergistic effect. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g. a compound of formula (I) and a combination partner, are both administered to a patient simultaneously in the form of a single entity or dosage. The term “non-fixed combination” means that the active ingredients, e.g. a compound of formula (I) and a combination partner, are both administered to a patient as separate entities either simultaneously, concurrently or sequentially with no, specific time limits, wherein such administration provides therapeutically effective levels of the two compounds in the body of the patient. The latter also applies to cocktail therapy, e.g., the administration of three or more active ingredients.

According to the invention, in a compound of formula (I) (“agent of the invention”), the following preferred, more preferred or particularly preferred aspects of the invention may be incorporated independently, collectively or in any combination:
R is preferably phenyl that is substituted by 1, 2 or 3 substituents selected from the group consisting of halogen, C₁-C₇-alkyl, trifluoromethyl, hydroxy and C₁-C₇-alkoxy. When R is phenyl substituted by C₁-C₇-alkyl it is preferably phenyl substituted by C₁-C₄-alkyl. When R is phenyl substituted by C₁-C₇-alkoxy it is preferably phenyl substituted by C₁-C₄-alkoxy.
R is more preferably phenyl that is substituted at one or two positions, especially two positions, by trifluoromethyl
R is particularly preferably 3,5-bis-trifluoromethyl-phenyl.
R¹ is preferably C₁-C₇-alkyl.
R¹ is more preferably C₁-C₄-alkyl.
R¹ is particular preferably methyl.
R² is preferably hydrogen or C₁-C₇-alkyl.
R² is particular preferably hydrogen.
R³ is preferably phenyl that is substituted by 1, 2 or 3 substituents selected from the group consisting of halogen, C₁-C₇-alkyl, trifluoromethyl, hydroxy and C₁-C₇-alkoxy,
R³ is more preferably phenyl that is substituted at one or two positions, especially two positions, by halo, especially chloro.
R³ is particularly preferably 3,4-dichloro-phenyl.
R⁵ is preferably D-azacycloheptan-2-on-3-yl.

In another embodiment of the invention there is provided a compound of formula (I) wherein
R is phenyl that is substituted by 1, 2 or 3 substituents selected from the group consisting of halogen, C₁-C₇-alkyl, trifluoromethyl, hydroxy and C₁-C₇-alkoxy;
R¹ is C₁-C₇-alkyl;
R² is hydrogen or C₁-C₇-alkyl;
R³ is phenyl that is substituted by 1, 2 or 3 substituents selected from the group consisting of halogen, C₁-C₇-alkyl, trifluoromethyl, hydroxy and C₁-C₇-alkoxy; and
R⁵ is D-azacycloheptan-2-on-3-yl,
   for the treatment of itch or an itch related disorder or disease, pruritus, or a dermatological disorder or disease as disclosed herein.

A particularly preferred compound of formula (I) has the chemical structure of formula (A)

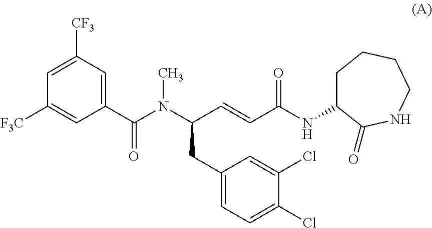

This compound is also known as N-[(E)-(R)-1-(3,4-Dichloro-benzyl)-3-((R)-2-oxo-azepan-3-ylcarbamoyl)-allyl]-N-methyl-3,5-bis-trifluoromethyl-benzamide or N—[(R,R)-(E)-1-(3,4-dichlorobenzyl)-3-(2-oxoazepan-3-yl)-carbamoyl]-allyl-N-methyl-3,5-bis(trifluoromethyl)-benzamide or (4R)-4-[N′-methyl-N′-(3,5-bis-trifluoromethyl-benzoyl)-amino]-4-(3,4-dichlorobenzyl)-but-2-enoic acid N—[(R)-epsilon-caprolactam-3-yl]-amide. The compound of formula (A), especially the hemihydrate thereof, is useful in the treatment of pruritus, or in the treatment of a dermatological disorder or disease. The compound of formula (A) is a pluripotent antagonist of the NK-1, NK-2 and NK-3 receptors, and according to the present invention the applicants have found that it is unexpectedly advantageous in the treatment of pruritus, or in the treatment of a dermatological disorder or disease. Particular advantages include one or more of the following: the treatment of chronic pruritus or a chronic dermatological disorder or disease, a particularly favourable efficacy, tolerability and/or side effect profile, and an anti-inflammatory effect, for example in the skin.

The compounds of formula (I) may be prepared by the processes generally and specifically described in WO98/07694 and WO2007/118551. In particular, the compound of formula (A) may be prepared using the process described in WO98/07694 Example 22, and in the Example on page 13 onwards of WO2007/118651.

The term “treatment” as used herein may include (1) therapeutic treatment, (it) prophylactic (preventive) treatment, (iii) delay of progression of a disorder or disease, (iv) curative treatment, (v) alleviation of a disorder or disease and/or (vi) alleviation of the symptoms of a disorder or disease.

The term “itch” is herein used interchangeably with the term pruritus and intended to have the same meaning.

The term “pruritus” is known to the person skilled in the art. It is a condition characterised by an unpleasant skin sensation, leading to the desire to scratch.

“Itch” or “pruritus” can be a symptom of many diseases, disease states, or disorders. It may also be present independently of a disease, disease state, or disorder. The term “itch” or “pruritus” includes itch, or pruritus, wherein the cause of the itch or pruritus is associated with or due to a disorder, disease or disease state, and includes itch or pruritus wherein the cause or origin is not understood.

The term “associated with” includes cases wherein both pruritus and the disorder or disease are present, and a link between them is suspected but not proven.

“Itch related disorder or disease” is known in the field. The term “itch related disorder or disease” means itch associated with or due to a disorder or disease. Accordingly, “itch related disorder or disease” means “pruritus related disorder or disease”, which means “pruritus associated with or due to a disorder or disease”.

“Disorder or disease” includes dermatological disease, systemic disease and neurological disorders.

The patient to be treated using the invention described herein is preferably a human. In an alternative embodiment, the invention provides the treatment of a non-human mammal, preferably a dog or cat,

“itch” or an “itch related disorder or disease”, particularly includes pruritoceptive itch, neurogenic itch, neuropathic itch, psychogenic itch and itch behaviors. More specifically, this includes pruritoceptive itch (originating in the skin, including itching arising from or associated with inflammatory skin diseases, e.g. skin diseases responsive to corticosteroid treatment and/or calcineurin inhibitor treatment, e.g. pimecrolimus, tacrolimus, cyclosporin A), neuropathic itch (due to a primary neurological disorder), neurogenic itch (arising from neurophysiological dysfunction) and idiopathic itch (itch of unknown cause e.g. idiopathic itch of the elderly (“senile pruritus” or chronic scalp itch).

Examples of itch associated with or due to such disorders or diseases include, but are not limited to the following, which are also embodiments of the present invention
(i) Pruritus associated with or due to a dermatological disorder or disease, in particular pruritic dermatoses. In particular, the dermatological disorder or disease is selected from atopic dermatitis, psoriasis, urticaria, irritant/allergic contact eczema/dermatitis, prurigo nodularis, insect bites, scabies, pediculosis, lichen ruber planus, folliculitis, dry skin (xerosis cubs), pruritus ani, pruritus scroti, pruritus vulvae and cutaneous T Cell Lymphoma (CTCL) (Sezary syndrome)
(ii) Pruritus associated with or due to metabolic disorders, including e.g. chronic renal disease, primary billiary cirrhosis, cholestasis, hepatic insufficieny, psychotropic medication;
(iii) Pruritus associated with or due to endocrine disorders, including e.g. thyrotoxicosis, hypothyroidism, hyperparathyroidism; hyperphosphatemia, diabetes mellitus;
(iv) Pruritus associated with or due to infectious diseases (e.g. chicken pox, fungal infections, post-herpes zoster, hepatitis C, tinea (pedis/corporis), pityriasis versicolor);
(v) Pruritus associated with or due to malignant and hematologic diseases, including e.g. lymphoma, leukemia myeloma, anemia, polycythemia vera;
(vi) Pruritus associated with or due to autoimmune diseases, including e.g. dermatitis herpetiformis, linear IgA syndrome, multiple sclerosis;
(vii) Pruritus associated with or due to medications, including e.g. adverse reactions;
(viii) Pruritus associated with or due to pregnancy (obstetric cholestasis, pemphigoid gestations, urticarial papules of pregnancy).

In an embodiment of the present invention, the disorder or disease is selected from the group consisting of pruritic dermatoses and pruritus due to metabolic disorders.

In another embodiment of the present invention, the disorder or disease is selected from the group consisting of pruritic dermatoses and pruritus due to endocrine disorders.

In another embodiment of the present invention, the disorder or disease is selected from the group consisting of pruritic dermatoses and pruritus due to infectious diseases.

In another embodiment of the present invention, the disorder or disease is selected from the group consisting of pruritic dermatoses and pruritus due to malignant and hematologic diseases.

In another preferred embodiment of the present invention, the disorder or disease is selected from the group consisting of pruritic dermatoses and pruritus due to autoimmune diseases.

In another embodiment of the present invention, the disorder or disease is selected from the group consisting of pruritic dermatoses and pruritus due to medications.

In another embodiment of the present invention, the disorder or disease is selected from the group consisting of pruritic dermatoses and pruritus due to pregnancy.

In a particular embodiment of the present invention, the disorder or disease is selected from the group consisting of pruritic dermatoses, more particularly from the group consisting of itch, atopic dermatitis, cutaneous T-cell lymphoma and psoriasis.

In one embodiment of the invention, there is provided a compound of formula (I), or a solvate or hydrate thereof as described herein, for the treatment of pruritus. The origin or cause of said pruritus may be known, uncertain or unknown, and the present invention includes the treatment of pruritis of any cause or origin. The invention also includes the treatment of pruritus associated with or due to more than one cause or origin, in the same patient. In another embodiment, the invention provides a treatment of chronic pruritus.

In another embodiment, the pruritus is associated with or due to a disorder or disease. In a further embodiment, the pruritus is associated with or due to a systemic disorder or disease. In a yet further embodiment, the pruritus is associated with or due to a neurological disorder or disease. In a different embodiment, the origin or cause of the pruritus is unknown.

In an embodiment of the invention, there is provided a compound of formula (I) or a solvate or hydrate thereof as described herein, for the treatment of pruritus due to or associated with a dermatological disease or disorder. In a particular embodiment of the invention, the pruritus is due to or associated with a pruritic dermatosis. In another embodiment, the pruritus is due to or associated with a disease or disorder selected from one or more of atopic dermatitis, psoriasis, urticaria, allergic contact eczema, allergic contact dermatitis, irritant contact dermatitis, irritant contact eczema, prurigo nodularis, insect bites, scabies, pediculosis, lichen ruber planus, folliculitis, dry skin (xerosis cutis), pruritus ani, pruritus scroti, pruritus vulvae and cutaneous T Cell Lymphoma (CTCL) (Sezary syndrome),

In a particular embodiment, there is provided the treatment of pruritus due to or associated with atopic dermatitis, psoriasis, cutaneous T-cell lymphoma, scabies or prurigo nodularis.

In another particular embodiment, there is provided the treatment of pruritus due to or associated with atopic dermatitis.

In another embodiment of the invention, there is provided a compound of formula (I), or a solvate or hydrate thereof as defined herein, for use in the treatment of a dermatological disorder or disease. In particular, the dermatological disorder or disease is selected from one or more of atopic dermatitis, psoriasis, urticaria, allergic contact eczema, allergic contact dermatitis, irritant contact dermatitis, irritant contact eczema, prurigo nodularis, insect bites, scabies, pediculosis, lichen ruber planus, folliculitis, dry skin (xerosis cutis), pruritus ani, pruritus scroti, pruritus vulvae and cutaneous T Cell Lymphoma (Sezary syndrome), and more particularly the dermatological disorder or disease is atopic dermatitis, psoriasis, scabies and prurigo nodularis. In a further embodiment, the disease is atopic dermatitis.

In another embodiment, the invention provides a compound of formula (I) or a solvate or hydrate thereof as defined herein, for use in the treatment of skin lesions, or pruritus associated with skin lesions.

For the above indications the appropriate dosage of the agents of invention will, of course, vary depending upon, for example, the host, the mode of administration and the nature and severity of the condition being treated as well as the relative potency of the particular agent of invention employed. Generally, the compound of formula (I) will be present in a therapeutically effective amount. For example, the amount of active agent required may be determined on the basis of known in vitro and in vivo techniques, determining how long a particular active agent concentration in the blood plasma remains at an acceptable level for a therapeutic effect. In general, satisfactory results in animals are indicated to be obtained at daily dosages of from about 0.01 to about 20.0 mg/kg p.o. In humans, an indicated daily dosage is in the range of from about 0.7 to about 1400 mg/day p.o., e.g. from about 10 to 200 mg, conveniently administered once or in divided doses up to 4× per day or in sustained release form. Oral dosage forms accordingly suitably comprise from about 0.2 or 2.0 to about 700 or 1400 mg agent of invention admixed with an appropriate pharmaceutically acceptable diluent or excipient therefore. In a particular embodiment, the dose is from 5 mg to 100 mg 2× per day.

For the treatment of a disease or disorder as described herein, any pharmaceutical formulation of a compound of formula (I) may be employed. Typical formulations for a compound of formula (I) are described in WO98/07694 e.g. in examples A to E, on pages 38 to 40. Other examples for such formulations comprising the agents of invention include, e.g. solid dispersions as disclosed in WO2008/077591 in examples 1 to 3, on pages 13 to 19, or microemulsions as disclosed in the examples in WO2005/074891 on pages 32 to 35. Preferably, a compound of formula (I) is provided in the form of an oral formulation, such as a tablet or a capsule. In a particular embodiment, the formulation is a solid dispersion. A compound of formula (I), or a combination as defined below, is typically present in an effective amount in such an oral formulation.

The contents of patent publications of publication numbers disclosed in the present application, are incorporated by reference in their entirety.

The treatment, as defined herein, is advantageously a systemic treatment, particularly an oral treatment.

In an embodiment, the invention provides a compound of formula (I) for the systemic treatment, particularly oral treatment, of itch or an itch related disorder or disease. Such treatment comprises administration to a patient of an effective amount of a compound of formula (I), as described above.

In accordance with the foregoing the present invention also provides:
(1) Use of a compound of formula (I), or a solvate or hydrate thereof as described herein, for the manufacture of a medicament for the treatment of pruritus, or an itch or an itch related disorder or disease. More particularly, the pruritus is of unknown or uncertain cause or origin, or is due to or associated with a disease or disorder as described herein.
   The invention also provides the use of a compound of formula (I), or a solvate or hydrate thereof as defined herein, for the manufacture of a medicament for the treatment of a dermatological disorder or disease, as described herein,
(2a) A method of treatment of pruritus, or an itch or itch related disorder or disease, comprising the step of administering to a subject in need thereof a therapeutically effective amount of a compound of formula (I), or a solvate or hydrate thereof as defined herein.
   In one embodiment, the pruritus is of unknown or uncertain cause or origin. In another embodiment, the pruritus is associated with or due to a disorder or disease. In a further embodiment, the pruritus is associated with or due to a dermatological disorder or disease. In a particular embodiment the pruritus is associated with or due to a dermatological disorder or disease selected from one or more of atopic dermatitis, psoriasis, urticaria, allergic contact eczema, allergic contact dermatitis, irritant contact dermatitis, irritant contact eczema, prurigo nodularis, insect bites, scabies, pediculosis, lichen tuber planus, folliculitis, dry skin (xerosis cutis), pruritus ani, pruritus scroti, pruritus vulvae and cutaneous T Cell Lymphoma (Sezary syndrome), particularly atopic dermatitis, psoriasis, scabies, cutaneous T-cell lymphoma, and prurigo nodularis.
(2b) A method of treatment of a dermatological disorder or disease comprising the step of administering to a subject in need thereof a therapeutically effective amount of a compound of formula (I), or a solvate or hydrate thereof, as defined herein. In particular, the dermatological disease or disorder is selected from one or more of atopic dermatitis, psoriasis urticaria, allergic contact eczema, allergic contact dermatitis, irritant contact dermatitis, irritant contact eczema, prurigo nodularis, insect bites, scabies, pediculosis, lichen rubes planus, folliculitis, dry skin (xerosis cutis), pruritus ani, pruritus scroti, pruritus vulvae and cutaneous T Cell Lymphoma (Sezary syndrome), more particularly atopic dermatitis, psoriasis, scabies, cutaneous T-cell lymphoma, and prurigo nodularis.
(3) Pharmaceutical composition, particularly oral formulation, comprising a compound of formula (I), or a solvate or hydrate thereof as described herein, and a pharmaceutically acceptable excipient, for the treatment of pruritus as described herein, or itch or an itch related disorder or disease, or a dermatological disorder or disease as described in (2a) or (2b) above.
(4) Use of a pharmaceutical composition, particularly an oral formulation, comprising a compound of formula (I) as active ingredient together with a pharmaceutically acceptable excipient, for the treatment as described in (2a) or (2b) above.
(5) A method of treatment as described in (2a) or (2b) above, comprising the step of administering to a subject in need thereof a pharmaceutical composition (particularly an oral formulation) comprising a therapeutically effective amount of a compound of formula (I), or a solvate or hydrate thereof as defined herein, and a pharmaceutically acceptable diluent or excipient.
(6) A combination which comprises (a) a compound of formula (I), or a solvate or hydrate thereof as defined herein, (b) a second drug substance or a pharmaceutically acceptable salt thereof and (c) optionally one or more pharmaceutically acceptable excipient(s) for the treatment as described in (2a) or (2b) above.

The second drug may be selected from ingredients which are known as being itch-reducers, such as e.g. emollients, topical campher and menthol, capsaicin, naltrexone, pramoxine, diphenylhydramine, anti-histamines, e g H1 blockers, caine anesthetics, e.g. benzocaine, cortisone, hydrocortisone or other corticosteroids, or it may be an anti-inflammatory agent such as e.g. pimecrolimus, tacrolirnus, cyclosporin A, diclofenac, ibuprofen, indomethacin, nabumetone, ketoprofen, naproxen, piroxicam, sulindac, etodolac meloxicam, rofecoxib, celecoxib, etoricoxib, parecoxib, valdecoxib, lumiracoxib and tilicoxib.

In a particular embodiment, the second drug is a topically administered drug. In a preferred embodiment, the inventive combination further contains one or more pharmaceutically acceptable excipient(s) as defined herein.

In a preferred embodiment, the inventive combination contains the compound of formula (I), or a solvate or hydrate thereof, and the second drug substance in a therapeutically effective amount. Such amount may be determined according to the methods described herein.

Another embodiment of the invention is directed to certain combinations disclosed herein that have a synergistic effect.

In accordance with the foregoing the present invention also provides:

(1) Use of a combination which comprises (a) a therapeutically effective amount of a compound of formula (I), or a solvate or hydrate thereof as defined herein (b) a second drug substance or a pharmaceutically acceptable salt thereof, (c) optionally one or more pharmaceutically acceptable excipient(s) for the manufacture of a medicament for the treatment of pruritus, or a dermatological disease, as described herein.

(2) A method of treatment of pruritus, or a dermatological disease comprising the step of administering to a subject in need thereof a therapeutically effective amount of a combination which comprises (a) a therapeutically effective amount of a compound of formula (I), or a solvate or hydrate thereof as defined herein, (b) a second drug substance or a pharmaceutically acceptable salt thereof and (c) optionally one or more pharmaceutically acceptable excipient(s).

The following examples illustrate the invention without limiting the scope thereof. In the examples provided below, the following abbreviations are used:
D day
ctb compared to baseline
BID twice a day
Test Compound compound of formula (A)

### EXAMPLE 1

### Oral Administration of Test Compound (Compound of Formula (A)) in Dogs with Flea Allergic Dermatitis

Flea-allergic laboratory Beagle dogs are infested with fleas. Fifty (50) Ctenocephalides felis fleas, 18 to 20 days old are released on the back of every dog on D1. Dogs are combed 48 hours (D3) after infestation to reduce the number of fleas (reduction of allergen charge).

Pruritus intensity: Pruritus intensity is evaluated using activity monitors (Actical™: Actiwatch™; Cambridge Neuorotechnology Ltd, Papworth Everard, UK) according to Nuttall and McEwan. (Nuttal T and McEwan N, “Objective measurement of pruritus in dogs: a preliminary study using activity monitors”. ESVD, 2006, 17 348-351). The physical activity monitors, mounted on collars, are placed around the dog's neck and tightened to the extent that they more with the skin but not so that they compress the underlying tissues. They were put on the dogs on D −5. The activity measured during the 3 to 4 nights prior to infestation with fleas shows the “normal” activity of each dog. This typically allows to see the difference in activity when the dogs have fleas and are developing pruritic and inflammatory lesions (i.e. dermatitis) and then again their activity during treatment with either the Test Compound or Prednisolone. The activity monitors are typically removed at the end of each phase on D12. Activity monitors measure the movement average within 15 seconds,

Skin observations: before flea infestations (D −1) all dogs are typically examined to establish a baseline score for erythema, papules, crusts, scales, alopecia, and excoriation. From D1, daily clinical observations are performed. Each lesion is typically graded using a scale from 0 to 3:

0=no signs
1=mild
2=moderate
3=severe

Skin lesions are allowed to develop until the total lesion score reaches ˜20. The Test Compound is then given orally at 50, 150 and 300 mg/day, divided into 2 doses, for 5 days. Prednisolone (10 mg/day, orally) is used as a comparator.

Erythema: is typically an acute clinical sign of cutaneous inflammation defined as redness of the skin caused by capillary congestion which can be aggravated by scratching. Excoriation: is typically a punctuate or linear abrasion produced by mechanical means, usually involving only the epidermis but not uncommonly reaching the papillary dermis. Alopecia; is typically the medical description of the loss of hair, usually induced by scratching (chewing or biting) in allergic dogs. Papule: is typically a circumscribed, solid elevation with no visible fluid, varying in size from a pinhead to 1 cm. crust, formed when there is a high break in the follicle wall. In the presence of bacteria, papules become pustules. Crusts: are typically dried serum, pus, or blood usually mixed with epithelial and sometimes bacterial debris. Scales: are dry or greasy laminated masses of keratin.

Lesion scores and Actical™ measurement values are typically observed on the following days:
Pre-value: before challenge with fleas
the day of challenge with fleas (Actical™ measurements only);
D1: the day when the total lesion score reached a sufficiently high value (average lesion score 20) and treatment of the animal started.
D2 to D6,

In the treatment group, lesion scores were taken from 8 dogs. Actical™ measurements were also taken from 6 of these dogs.

In the untreated group, Actical™ measurements and lesion scores were taken from 3 dogs. In the non-allergic group, Actical™ measurements were taken from 2 dogs and lesion scores from 3 dogs.

Example 1a: Pruritus - Daytime activity - in flea allergic dogs

| | | Means (standard deviations) | | | | | |
|---|---|---|---|---|---|---|---|
| | | | H: Test | M: Test | L: Test | | |
| | | P: | Compound | Compound | Compound | U: | Non- |
| response | day | Prednisolone | 300 mg | 150 mg | 50 mg | untreated | responders |
| Actical | (P) pre | 40 (10) | 58 (21) | 31 (8) | 46 (16) | 43 (6) | 27 (2) |
| day** | (F) fleas | 74 (22) | 93 (37) | 53 (16) | 84 (47) | 69 (20) | 37 (0) |
| | 1 | 74 (20) | 114 (35) | 58 (17) | 88 (46) | 106 (27) | 21 (0) |
| | 2 | 66 (50) | 116 (77) | 48 (13) | 64 (40) | 149 (14) | 23 (3) |
| | 3 | 49 (33) | 99 (38) | 53 (15) | 72 (35) | 162 (24) | 24 (2) |
| | 4 | 54 (22) | 107 (28) | 44 (17) | 67 (37) | 194 (8) | 23 (2) |
| | 5 | 47 (24) | 129 (53) | 60 (41) | 62 (40) | 247 (51) | 21 (2) |
| | 6 | 41 (14) | 79 (21) | 40 (21) | 48 (31) | 239 (15) | 23 (12) |

*D1: when animals reached a sufficient lesion score to initiate treatment, Pre: pre-values, before exposure to fleas
**Average activity counts every 15 secs (6am-6pm)
A graphical representation of the data (mean) is shown in FIG. 1

The above data show the activity control back to baseline after treatment, while untreated control animals experience a significant increase in activity. All Test Compound doses control pruritic activity. In particular, 150 mg and 50 mg are equipotent to the Prednisolone controls. This is demonstrated by the reduction of values of activity from day 1 to close to that recorded at baseline after 5 days of BID administrations

Example 1b: Pruritus - Nighttime activity - in flea allergic dogs

| | | Means (standard deviations) | | | | | |
|---|---|---|---|---|---|---|---|
| | | | H: Test | M: Test | L: Test | | |
| | | P: | Compound | Compound | Compound | U: | Non- |
| response | day | Prednisolone | 300 mg | 150 mg | 50 mg | untreated | responders |
| Actical | Pre | 7.9 (4.4) | 7.0 (3.8) | 4.2 (2.2) | 5.2 (3.3) | 8.5 (2.2) | 5.2 (1.4) |
| night** | Fleas | 8.6 (3.5) | 6.8 (3.0) | 6.2 (3.8) | 6.8 (4.9) | 8.9 (2.7) | 5.8 (1.1) |
| | 1* | 16.2 (2.9) | 24.6 (22.5) | 14.6 (4.2) | 22.1 (13.5) | 15.0 (3.0) | 3.9 (1.4) |
| | 2 | 6.8 (4.1) | 11.9 (4.8) | 10.7 (6.8) | 13.2 (11.8) | 19.0 (2.6) | 5.3 (1.8) |
| | 3 | 5.1 (2.8) | 16.8 (9.5) | 10.6 (7.5) | 11.5 (7.3) | 18.5 (2.1) | 3.5 (1.1) |
| | 4 | 7.3 (3.3) | 20.7 (10.5) | 9.4 (4.2) | 12.6 (9.9) | 21.9 (3.5) | 3.2 (1.1) |
| | 5 | 4.8 (2.2) | 16.2 (13.2) | 10.4 (6.5) | 9.8 (6.8) | 26.7 (3.3) | 3.5 (0.6) |
| | 6 | 5.3 (1.7) | 13.3 (10.2) | 11.2 (7.4) | 9.9 (7.4) | 29.8 (2.5) | 4.0 (0.4) |

*D1: when animals reached a sufficient lesion score to initiate treatment. Pre: pre-values, before exposure to fleas
**Average activity counts every 15 secs (6pm-6am)
A graphical representation of the data (mean) is shown in FIG. 2.

The above data depict the positive efficacy of Test Compound doses (in particular 50 mg) in controlling activity back to baseline from day 1 of BID administrations, while the activity scores for untreated control animals continue to rise. Nighttime activity is considered to be a better indicator for pruritus intensity than daytime activity, since the animals are asleep and not distracted.

Example 1c: Flea-allergic dermatitis in dogs (total lesion scores)

| | | Means (standard deviations) | | | | | |
|---|---|---|---|---|---|---|---|
| | | | H: Test | M: Test | L: Test | | |
| | | P: | Compound | Compound | Compound | U: | Non- |
| response | day | Prednisolone | 300 mg | 150 mg | 50 mg | untreated | responders |
| Total | 2 | −5.6 (6.3) | −3.6 (9.1) | −2.3 (3.3) | −3.7 (4.9) | 2.0 (1.8) | −0.5 (0.7) |
| score ctb | 3 | −7.9 (6.7) | −4.1 (6.4) | −3.9 (4.0) | −2.8 (5.5) | 3.7 (2.3) | −1.0 (1.4) |
| | 4 | −9.2 (6.3) | −7.6 (8.4) | −6.4 (3.3) | −1.9 (7.1) | 6.0 (3.0) | −0.8 (1.1) |
| | 5 | −9.9 (4.8) | −7.6 (9.7) | −5.0 (3.8) | −2.8 (6.4) | 10.7 (3.3) | −0.5 (0.7) |
| | 6 | −11.8 (6.1) | −6.7 (8.1) | −8.8 (8.8) | −8.7 (9.9) | 10.3 (1.5) | −0.5 (0.7) |

### Score Including all Lesions:

Erythema*,
Excoriations*,
Papules/pustules
Self induced alopecia^{o}, and
Scales,
Erythema and excoriations are related to pruritus
Self induced alopecia is also related to pruritus but the improvement is not observed after several weeks of treatment.

Day 1 of treatment when dogs reached a sufficient lesion score to initiate treatment (average 20). Pre-values (baseline).

A graphical representation of the data with the lesion score on Day 1 set to 100% for each group is shown in FIG. 3. (Data points are depicted as mean + or − standard deviations.)

The above data show the reduction of the lesion score, i.e. the improvement after treatment with Test Compound 300 mg, 150 mg, 50 mg compared to dogs left untreated and to normal non-flea allergic dogs. The inhibition achieved with the Test Compound is in the same range as the potent corticosteroid prednisolone.

The results indicate that the Test Compound exerts significant effects both in controlling pruritus and inflammatory skin lesions in dogs with flea allergic dermatitis. Doses between 50 mg to 150 mg Test Compound (5 to 15 mg/kg for a 10 kg dog) are particularly effective.

Such efficacy for the pluripotent NK antagonist test compound of formula (A) could not have been predicted and is one advantage of the present invention. In particular, the results show significant efficacy on both the pruritic component and the inflammatory component of the dermatitis. Surprisingly, the efficacy of the compound of formula (A) is similar to that of the potent oral corticosteroid prednisolone herein used as a positive control. Furthermore, said effects are advantageously demonstrated over several days, showing a beneficial effect for chronic pruritus, or chronic diseases or disorders.

1.-13. (canceled)

14. A method of treatment of pruritus comprising the step of administering to a subject in need thereof, a therapeutically effective amount of a compound of formula (I), 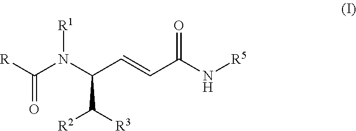
wherein
R is phenyl that is unsubstituted or is substituted by 1, 2 or 3 substituents selected from the group consisting of halogen, C1-C7-alkyl, trifluoromethyl, hydroxy and C1-C7-alkoxy;
R1 is hydrogen or C1-C7-alkyl;
R2 is hydrogen, C1-C7-alkyl or phenyl that is unsubstituted or is substituted by 1, 2 or 3 substituents selected from the group consisting of halogen, C1-C7-alkyl, trifluoromethyl, hydroxy and C1-C7-alkoxy;
R3 is phenyl that is unsubstituted or is substituted by 1, 2 or 3 substituents selected from the group consisting of halogen, C1-C7-alkyl, trifluoromethyl, hydroxy and C1-C7-alkoxy, or R3 is naphthyl, 1H-indol-3-yl or 1-C1-C7-alkyl-indol-3-yl; and
R5 is C3-C8-cycloalkyl, D-azacycloheptan-2-on-3-yl or L-azacycloheptan-2-on-3-yl;
or a solvate or hydrate thereof.

15. A method of treatment of pruritus comprising the step of administering to a subject in need thereof, a therapeutically effective amount of N-[(E)-(R)-1-(3,4-Dichloro-benzyl)-3-((R)-2-oxo-azepan-3-ylcarbamoyl)-allyl]-N-methyl-3,5-bis-trifluoromethyl-benzamide or the hemihydrate thereof.

16. A method of treatment of pruritus according to claim 14, wherein the pruritus is of unknown or uncertain cause or origin, or is associated with or due to a disorder or disease.

17. A method of treatment of pruritus according to claim 14, wherein the pruritus is associated with or due to a dermatological disorder or disease.

18. A method of treatment of pruritus according to claim 14, wherein the pruritus is associated with or due to a dermatological disease or disorder selected from one or more of atopic dermatitis, psoriasis, urticaria, allergic contact eczema, allergic contact dermatitis, irritant contact dermatitis, irritant contact eczema, prurigo nodularis, insect bites, scabies, pediculosis, lichen ruber planus, folliculitis, dry skin (xerosis cutis), pruritus ani, pruritus scroti, pruritus vulvae and cutaneous T Cell Lymphoma (Sezary syndrome).

19. A method of treatment of pruritus according to claim 14, wherein the pruritus is associated with or due to a dermatological disease or disorder selected from one or more of atopic dermatitis, psoriasis, scabies, cutaneous T-cell lymphoma, and prurigo nodularis.

20. A method of treatment of a dermatological disorder or disease comprising the step of administering to a subject in need thereof a therapeutically effective amount of a compound of formula (I), 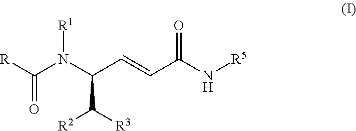
wherein
R is phenyl that is unsubstituted or is substituted by 1, 2 or 3 substituents selected from the group consisting of halogen, C1-C7-alkyl, trifluoromethyl, hydroxy and C1-C7-alkoxy;
R1 is hydrogen or C1-C7-alkyl;
R2 is hydrogen, C1-C7-alkyl or phenyl that is unsubstituted or is substituted by 1, 2 or 3 substituents selected from the group consisting of halogen, C1-C7-alkyl, trifluoromethyl, hydroxy and C1-C7-alkoxy;
R3 is phenyl that is unsubstituted or is substituted by 1, 2 or 3 substituents selected from the group consisting of halogen, C1-C7-alkyl, trifluoromethyl, hydroxy and C1-C7-alkoxy, or R3 is naphthyl, 1H-indol-3-yl or 1-C1-C7-alkyl-indol-3-yl; and
R5 is C3-C8-cycloalkyl, D-azacycloheptan-2-on-3-yl or L-azacycloheptan-2-on-3-yl;
or a solvate or hydrate thereof.

21. A method of treatment as claimed in claim 20, wherein the dermatological disorder or disease is selected from one or more of atopic dermatitis, psoriasis, urticaria, allergic contact eczema, allergic contact dermatitis, irritant contact dermatitis, irritant contact eczema, prurigo nodularis, insect bites, scabies, pediculosis, lichen ruber planus, folliculitis, dry skin, pruritus ani, pruritus scroti, pruritus vulvae and cutaneous T Cell Lymphoma.

22. A method of treatment as claimed in claim 21, wherein the dermatological disorder or disease is selected from one or more of atopic dermatitis, psoriasis, scabies, cutaneous T-cell lymphoma, and prurigo nodularis.

23-26. (canceled)

27. A method of treatment of pruritus associated with or due to a dermatological disorder or disease selected from one or more of atopic dermatitis, psoriasis, scabies, cutaneous T-cell lymphoma, and prurigo nodularis, comprising the step of administering to a subject in need thereof, a therapeutically effective amount of N-[(E)-(R)-1-(3,4-Dichloro-benzyl)-3-((R)-2-oxo-azepan-3-ylcarbamoyl)-allyl]-N-methyl-3,5-bis-trifluoromethyl-benzamide or the hemihydrate thereof.

28. A method of treatment of a dermatological disorder or disease selected from one or more of atopic dermatitis, psoriasis, scabies, cutaneous T-cell lymphoma, and prurigo nodularis, comprising the step of administering to a subject in need thereof, a therapeutically effective amount of N-[(E)-(R)-1-(3,4-Dichloro-benzyl)-3-((R)-2-oxo-azepan-3-ylcarbamoyl)-allyl]-N-methyl-3,5-bis-trifluoromethyl-benzamide or the hemihydrate thereof.

29. The method of claim 14, wherein
R is phenyl that is substituted by 1, 2 or 3 substituents selected from the group consisting of halogen, C1-C7-alkyl, trifluoromethyl, hydroxy and C1-C7-alkoxy;
R1 is or C1-C7-alkyl;
R2 is hydrogen or C1-C7-alkyl;
R3 is phenyl that is substituted by 1, 2 or 3 substituents selected from the group consisting of halogen, C1-C7-alkyl, trifluoromethyl, hydroxy and C1-C7-alkoxy; and
R5 is D-azacycloheptan-2-on-3-yl.

30. The method of claim 15, wherein the pruritus is of unknown or uncertain cause or origin, or is associated with or due to a disorder or disease.

31. The method of claim 15, wherein the pruritus is associated with or due to a dermatological disorder or disease.

32. The method of claim 15, wherein the pruritus is associated with or due to a dermatological disease or disorder selected from one or more of atopic dermatitis, psoriasis, urticaria, allergic contact eczema, allergic contact dermatitis, irritant contact dermatitis, irritant contact eczema, prurigo nodularis, insect bites, scabies, pediculosis, lichen ruber planus, folliculitis, dry skin, pruritus ani, pruritus scroti, pruritus vulvae and cutaneous T Cell Lymphoma (Sezary syndrome).

33. The method of claim 15, wherein the pruritus is associated with or due to a dermatological disease or disorder selected from one or more of atopic dermatitis, psoriasis, scabies, cutaneous T-cell lymphoma, and prurigo nodularis.
